(19) United States
(12) Patent Application Publication
(10) Pub. No.: US 2017/0044506 A1
(43) Pub. Date: Feb. 16, 2017

(54) cDNA SYNTHESIS USING A REVERSIBLY INACTIVATED REVERSE TRANSCRIPTASE

(71) Applicant: LIFE TECHNOLOGIES CORPORATION, Carlsbad, CA (US)

(72) Inventors: Lei (Larry) XI, Foster City, CA (US); Roland Nagel, Santa Cruz, CA (US); Stephen Hendricks, Los Gatos, CA (US); Jennifer Berkman, San Francisco, CA (US); Marian Peris, San Mateo, CA (US); Yulei Wang, Foster City, CA (US)

(21) Appl. No.: 15/243,808

(22) Filed: Aug. 22, 2016

Related U.S. Application Data
(62) Division of application No. 12/900,794, filed on Oct. 8, 2010, now abandoned.
(60) Provisional application No. 61/250,478, filed on Oct. 9, 2009.

(51) Int. Cl. C12N 9/12; C12N 15/10

(52) U.S. Cl. CPC C12N 9/1276; C12Y 207/07049; C12N 15/1096

(57) ABSTRACT

The present invention provides compositions and methods for a reverse transcription reaction using a reversibly inactivated reverse transcriptase enzyme. The reversibly inactivated reverse transcriptase enzyme results from a chemical modification which inactivates the reverse transcriptase enzyme. The activity of the reverse transcriptase enzyme is recovered by an incubation of the reaction mixture at elevated temperature prior to, or as part of the reverse transcription reaction. The reverse transcriptase enzyme of the present invention provides for a significant reduction in non-specific reverse transcription from template nucleic acid molecules because the formulation of the reaction mixture does not support the formation of reverse transcription products prior to activation of the reverse transcriptase.

### RELATED APPLICATIONS

This application is a divisional application of U.S. application Ser. No. 12/900,794, filed Oct. 8, 2010, which claims the benefit of U.S. Provisional Application No. 61/250,478, filed Oct. 9, 2009, the contents of which are incorporated herein in their entirety.

### SEQUENCE LISTING

The instant application contains a Sequence Listing disclosed in a paper copy of the sequence listing for the present application. The paper copy of the sequence listing and the computer readable copy are the same and do not introduce new matter.

### FIELD OF THE INVENTION

This invention relates generally to the field of nucleic acid chemistry. More specifically, it relates to methods of reverse transcription by reversibly inactivated reverse transcriptase enzymes and more specifically to methods for the reducing non-specific reverse transcriptase activity.

### BACKGROUND

A common technique used to study gene expression in living cells is to the produce a DNA copy (cDNA) of the cellular complement of RNA. This technique provides a means to study RNA from living cells which avoids the direct analysis of inherently unstable RNA. As a first step in cDNA synthesis, the RNA molecules from an organism are isolated from an extract of cells or tissues of the organism. After mRNA isolation, using methods such as affinity chromatography utilizing oligo dT, oligonucleotide sequences are annealed to the isolated mRNA molecules and enzymes with reverse transcriptase activity can be utilized to produce cDNA copies of the RNA sequence, utilizing the RNA/DNA primer as a template. Thus, reverse transcription of mRNA is a key step in many forms of gene expression analyses. Generally, mRNA is reverse transcribed into cDNA for subsequent analysis by primer extension or polymerase chain reaction.

The reverse transcription of RNA templates requires a primer sequence which is annealed to an RNA template in order for DNA synthesis to be initiated from the 3′ OH of the primer. While not operating at their optimal temperatures, reverse transcriptase enzymes are active at room temperature. At these lower temperatures, primers may form both perfectly matched as well as mismatched DNA/RNA hybrids. Under these conditions, reverse transcriptase is capable of extending from perfectly matched primer/template complexes as well as from mismatched primer sequences at room temperature. In some instances, a reverse transcriptase enzyme can produce large amounts of non-specific cDNA products as a result of such non-specific priming events. The products of non-specific reverse transcription can interfere with subsequent cDNA analyses, such as cDNA sequencing, real-time PCR, and alkaline agarose gel electrophoresis, among others. Non-specific cDNA templates produced by non-specific reverse transcriptase activity can present particular difficulties in applications such as real-time PCR. In particular, such non-specific cDNA products can give rise to false signals which can complicate the analysis of real-time PCR signals and products. Thus, the reduction of non-specific reverse transcriptase activity would result in greater specificity of cDNA synthesis. Currently, there are no reliable and easy to use methods for the improving the specificity of reverse transcription. The present invention satisfies these and other needs.

### SUMMARY

The present invention provides methods and reagents for reverse transcribing a nucleic acid molecule nucleic using a primer-based reverse transcription reaction which provides a simple and economical solution to the problem of non-specific reverse transcription. The methods use reversibly inactivated reverse transcriptase enzymes which can be reactivated by incubation in the reverse transcription reaction mixture at an elevated temperature. Non-specific reverse transcription is greatly reduced because the reaction mixture does not support primer extension until the temperature of the reaction mixture has been elevated to a temperature which improves primer hybridization specificity.

Accordingly, one embodiment of the present invention provides a modified reverse transcriptase enzyme, in which, the modified reverse transcriptase enzyme is produced by the reaction of a mixture of a reverse transcriptase enzyme which catalyzes a primer extension reaction and a modifier reagent, in which, the reaction results in a covalent chemical modification of the enzyme which results in inactivation of enzyme activity, and in which, incubation of the modified enzyme in an aqueous buffer under non-activating conditions results in no significant increase in reverse transcriptase enzyme activity, and, in which, incubation of said modified enzyme in an aqueous buffer under activating conditions results in an increase in enzyme activity.

Another embodiment of the present invention provides a method for the reverse transcription of a target nucleic acid contained in a sample with the steps of: (a) contacting the sample with a reverse transcription reaction mixture containing a primer complementary to the target nucleic acid and a modified reverse transcriptase enzyme, in which, the modified reverse transcriptase enzyme is produced by the reaction of a mixture of a reverse transcriptase enzyme which catalyzes a primer extension reaction and a modifier reagent, in which, the reaction results in a covalent chemical modification of the reverse transcriptase enzyme which results in inactivation of enzyme activity, in which, incubation of said modified reverse transcriptase enzyme in an aqueous buffer under non-activating conditions results in no significant increase in enzyme activity, and wherein incubation of the modified enzyme in an aqueous buffer under activating conditions results in an increase in enzyme; and (b) incubating the resulting mixture of step (a) under activating conditions for a time sufficient to reactivate said reverse transcriptase enzyme and allow formation of primer extension products.

In various aspects of the above embodiments, the non-activating conditions comprise alkaline pH at a temperature less than about 25° C., and the activating conditions can include subjecting the enzyme formulated at about pH 6.5-9 at 25° C. to a temperature greater than about 40° C. In some embodiments the temperature may be 40° C. to 50° C., 50° C. to 60° C., or 60° C. to 65° C.

In other aspects of the above embodiments, there is no significant increase in reverse transcriptase enzyme activity in less than about 20 minutes.

In yet other aspects of the above embodiments, the increase in enzyme activity is at least two-fold, and the increase in enzyme activity occurs in less than about 60 minutes.

In further aspects of the above embodiments, the modifier can be maleic anhydride; exo-cis-3,6-endoxo-Δ⁴-tetrahydrophthalic anhydride; citraconic anhydride; 3,4,5,6-tetrahydrophthalic anhydride; cis-aconitic anhydride; and 2,3-dimethylmaleic anhydride. In some favorable aspects, the modifier reagent is 2,3-dimethylmaleic anhydride.

In yet further aspects of the above embodiments, the modified reverse transcriptase can have an inactivation that is at least 50%, 60%, 70%, 80%, or 90%. In some exemplary aspects, the inactivation is essentially complete.

In a further embodiment, the present invention provides a modified reverse transcriptase enzyme, in which, the modified reverse transcriptase enzyme is produced by the reaction of a mixture of a reverse transcriptase enzyme which catalyzes a primer extension reaction and a modifier reagent, in which, the reaction results in a covalent chemical modification of the enzyme which results in essentially complete inactivation of enzyme activity, in which, incubation of said modified enzyme in an aqueous buffer at alkaline pH at a temperature less than about 25° C. results in no significant increase in reverse transcriptase enzyme activity in less than about 20 minutes, and in which, incubation of said modified enzyme in an aqueous buffer, formulated to about pH 6.5-9 at 25° C., at a temperature greater than about 40° C. results in at least a two-fold increase in enzyme activity in less than about 60 minutes.

A yet further embodiment provides a method for the reverse transcription of a target nucleic acid contained in a sample including the steps of: (a) contacting the sample with a reverse transcription reaction mixture containing a primer complementary to the target nucleic acid and a modified reverse transcriptase enzyme, in which, the modified reverse transcriptase enzyme is produced by a reaction of a mixture of a reverse transcriptase enzyme which catalyzes a primer extension reaction and a modifier reagent, in which, the reaction results in a covalent chemical modification of the reverse transcriptase enzyme which results in essentially complete inactivation of enzyme activity, in which, incubation of the modified reverse transcriptase enzyme in an aqueous buffer at alkaline pH at a temperature less than about 25° C. results in no significant increase in enzyme activity in less than about 60 minutes, and in which, incubation of the modified enzyme in an aqueous buffer, formulated to about pH 6.5-9 at 25° C., at a temperature greater than about 40° C. results in at least a two-fold increase in enzyme activity in less than about 60 minutes; and (b) incubating the resulting mixture of step (a) at a temperature which is greater than about 40° C. for a time sufficient to reactivate said reverse transcriptase enzyme and allow formation of primer extension products. In some embodiments the incubation temperature may be 40° C. to 50° C., 50° C. to 60° C., or 60° C. to 65° C.

Other embodiments provide a method for strand specific reverse transcription of a target nucleic acid in a sample comprising sense and antisense transcription products comprising (a) contacting the sample with a reverse transcription reaction mixture containing a primer complementary to one of the sense or antisense transcription products and a modified reverse transcriptase enzyme, wherein the modified reverse transcriptase enzyme is produced by a reaction of a mixture of a reverse transcriptase enzyme which catalyzes a primer extension reaction and a modifier reagent, wherein the reaction results in a covalent chemical modification of the reverse transcriptase enzyme which results in inactivation of enzyme activity, wherein incubation of the modified reverse transcriptase enzyme in an aqueous buffer under non-activating conditions results in no significant increase in enzyme activity, wherein incubation of the modified enzyme in an aqueous buffer under activating conditions results in an increase in enzyme activity; and (b) incubating the resulting mixture of step (a) under activating conditions for a time sufficient to reactivate the reverse transcriptase enzyme and allow formation of primer extension products.

In various aspects of the embodiments, the modifier reagent can include maleic anhydride; exo-cis-3,6-endoxo-Δ⁴-tetrahydrophthalic anhydride; citraconic anhydride; 3,4,5,6-tetrahydrophthalic anhydride; cis-aconitic anhydride; and 2,3-dimethylmaleic anhydride. In some favorable aspects, the modifier reagent is 2,3-dimethylmaleic anhydride.

A further embodiment of the present invention provides kits for carrying out a reverse transcription reaction including a modified reverse transcriptase enzyme as described in the embodiments and aspects described above.

It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the disclosure and together with the description, serve to explain certain teachings.

### BRIEF DESCRIPTION OF THE DRAWINGS

The skilled artisan will understand that the drawings described below are for illustration purposes only. The drawings are not intended to limit the scope of the present teachings in any way.

FIG. 1 shows the structures of 2,3-dimethylmaleic anhydride, and the reaction between 2,3-dimethylmaleic anhydride and lysine.

FIG. 2 shows the results of an activity assay for 2,3-dimethylmaleic anhydride-modified RQ1 reverse transcriptase and unmodified RQ1 reverse transcriptase.

FIG. 3 shows the results of an activity assay for the reactivation of 2,3-dimethylmaleic anhydride modified RQ1 reverse transcriptase with increasing levels of 2,3-dimethylmaleic anhydride modification.

FIG. 4 shows the results of a RT-PCR specificity assay using 2,3-dimethylmaleic anhydride-modified RQ1 reverse transcriptase.

FIG. 5 shows the results of a RT-PCR specificity assay using unmodified RQ1 reverse transcriptase.

FIG. 6 shows the results of reactivation of 2,3-dimethylmaleic anhydride modified SuperScript® III reverse transcriptase using a reverse transcriptase activity.

FIG. 7 shows the bacterial plasmid pBluescript II KS+ with five bacterial gene inserts: BioB, BioC, BioD, Lys and Phe.

FIG. 8 shows the dynamic range and sensitivity of the strand-specific assay over 7-log of in vitro transcribed bacterial transcripts in either sense or anti-sense strand using Multiscript, HS-RQ1 (SSTAY), ThermoScript™, SuperScript® III and rTth reverse transcriptases.

FIG. 9 shows the accuracy of strand-specific assays by varying the ratio of sense to antisense transcripts over a 1000 fold range when using the HS-RQ1 (SSTAY), SuperScript® III and ThermoScript™ reverse transcriptases.

FIG. 10 shows the mammalian plasmid pCMV6-XL4/5/6 with eight mammalian full length cDNA inserts and PCR strategy to introduce T7 promoter into these gene constructs to produce in vitro transcribed full length mammalian RNA transcripts.

FIG. 11 shows the specificity of the strand-specific assays by measuring the delta Ct (dCt) of the sense versus the antisense strands using Multiscript, Qiagen Q-Script, rTth and HS-RQ1HS (SSTAY) reverse transcriptases.

### DETAILED DESCRIPTION

### I. Introduction

The specificity of reverse transcription depends on the specificity of primer hybridization to a target RNA sequence. Primers may be selected to be complementary to, or substantially complementary to, sequences occurring at the 3′ end of each strand of the target nucleic acid sequence. At the temperatures used in a typical reverse transcriptase reaction, the primers may hybridize to many non-target sequences as well as the intended target sequence. Additionally, reverse transcription reaction mixtures are typically assembled at room temperature, well below the temperature needed to insure specific primer hybridization. Under such less stringent conditions, primers may bind non-specifically to partially complementary RNA sequences (or even to other primers) and initiate the synthesis of undesired extension products, which can be reverse transcribed along with the correct target sequence, resulting in the production non-specific cDNA. Non-specific cDNA extension products can compete with the specific cDNA reverse transcriptase products in later applications. For example, the presence of non-specific cDNA extension products can significantly decrease the efficiency of the detection of specific cDNA products in RT-PCR. Thus, in these instances it would be highly advantageous to be able to reverse transcribe RNA templates at temperatures which preclude the formation of non-specific primer template complexes. Presently, there are no known reverse transcriptase enzymes which can be activated at temperatures sufficient to prevent non-specific reverse transcription from non-specific primer/template complexes. Therefore, there is a need for reverse transcriptase enzymes that can be activated at elevated temperatures that inhibit the formation of non-specific primer/templates.

Several methods exist to address the problem of non-specific amplification products that arise from non-specific extension by thermostable DNA polymerases during PCR. In the case of PCR, non-specific products are caused by the extension of misprimed oligonucleotides during the reaction set-up or the initial heating phase of a PCR reaction, and essential components such as the oligonucleotide primers, nucleotide triphosphates, magnesium ions, or thermostable nucleic acid polymerases are sequestered for release at higher temperatures, thereby reducing the probability of having non-specific hybridization or the extension of misprimed oligonucleotides. These techniques are referred to as “manual hot-start PCR” methods. Another method for reducing formation of extension products from misprimed oligonucleotides during a PCR reaction set-up entails the use of a reversible chemically modified thermostable DNA polymerase that becomes active only after incubation at an elevated temperature, thus preventing the production of non-specific DNA synthesis during reaction set-up and the initial heating phase of PCR. U.S. Pat. Nos. 5,677,152 and 5,773,258, and corresponding European patent publication EP 0771 870 A1 describe a method for the amplification of a target nucleic acid using a thermostable polymerase reversibly inactivated using dicarboxylic acid anhydride compounds. However there is no known method to easily and reliably control non-specific reverse transcription resulting from mismatched primer sequences. In many instances it would be desirable to initiate reverse transcription reactions at temperatures, above which, the formation of non-specific primer complexes is inhibited.

Accordingly, the present invention provides compositions and methods for reverse transcribing a nucleic acid molecule nucleic using a primer-based reverse transcription reaction which provides a simple and economical solution to the problem of non-specific reverse transcription. The methods disclosed herein use reversibly inactivated reverse transcriptase enzymes which can be reactivated by incubation in the reverse transcription reaction mixture at an elevated temperature. Non-specific reverse transcription is greatly reduced because the reaction mixture does not support primer extension until the temperature of the reaction mixture has been elevated to a temperature which improves primer hybridization specificity. Reduced non-specific reverse transcription may also allow for the selective transcription of either the sense or antisense transcript from a biological sample containing both transcripts.

Specifically, the present disclosure relates to reversibly inactivated reverse transcriptase enzymes which are produced by a reaction between a reverse transcriptase enzyme and a modifier reagent. The reactions disclosed herein result in a significant, and preferably essentially complete, reduction in reverse transcriptase enzyme activity at low temperature (i.e., non-activating conditions). As discussed in greater detail herein, the present inventors have generated modified reverse transcriptase enzymes through the reaction of a reverse transcriptase enzyme and a dicarboxylic acid anhydride of the general formula:

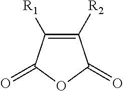

where R1 and R2 are hydrogen or organic radicals, which may be linked, or of the general formula:

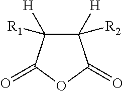

where R1 and R2 are organic radicals, which may be linked, and the hydrogen are cis. The reactions disclosed herein result in essentially complete inactivation of enzyme activity at ambient temperatures, such as those used to set-up reverse transcription reactions, and restoration of activity upon exposure to higher temperatures that inhibit the formation of mismatched primer/template complexes.

### II. Definitions

For the purposes of interpreting of this specification, the following definitions will apply, and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The term “hybridization” refers generally to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which only fully complementary nucleic acid strands will hybridize are referred to as “stringent hybridization conditions” or “sequence-specific hybridization conditions”. Stable duplexes of substantially complementary sequences can be achieved under less stringent hybridization conditions. Those skilled in the art of nucleic acid technology can determine duplex stability empirically considering a number of variables including, for example, the length and base pair concentration of the oligonucleotides, ionic strength, and incidence of mismatched base pairs, following the guidance provided by the art (see, e.g., Sambrook et al., 1989, supra). Generally, stringent hybridization conditions are selected to be about 5° C. lower than the thermal melting point (Tm) for the specific sequence at a defined ionic strength and pH. The Tm is the temperature (under defined ionic strength and pH) at which 50% of the base pairs have dissociated. Relaxing the stringency of the hybridization conditions will allow sequence mismatches to be tolerated; the degree of mismatch tolerated can be controlled by suitable adjustment of the hybridization conditions.

The term “primer” refers generally to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature.

A primer is preferably a single-stranded oligodeoxyribonucleotide, although oligonucleotide analogues, such as “peptide nucleic acids”, can act as primers and are encompassed within the meaning of the term “primer” as used herein. The appropriate length of a primer depends on the intended use of the primer, but typically ranges from 6 to 50 nucleotides. Short primer molecules generally require cooler temperatures to form sufficiently stable hybrid complexes with the template. A primer need not reflect the exact sequence of the template nucleic acid, but must be sufficiently complementary to hybridize with the template.

The term “primer extension” as used herein refers to both to the synthesis of DNA resulting from the polymerization of individual nucleoside triphosphates using a primer as a point of initiation, and to the joining of additional oligonucleotides to the primer to extend the primer. Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning of the amplified product. The region of the primer which is sufficiently complementary to the template to hybridize is referred to herein as the hybridizing region. The terms “target region” and “target nucleic acid” refers to a region or subsequence of a nucleic acid which is to be reverse transcribed.

The primer hybridization site can be referred to as the target region for primer hybridization. As used herein, an oligonucleotide primer is “specific” for a target sequence if the number of mismatches present between the oligonucleotide and the target sequence is less than the number of mismatches present between the oligonucleotide and non-target sequences which may be present in the sample. Hybridization conditions can be chosen under which stable duplexes are formed only if the number of mismatches present is no more than the number of mismatches present between the oligonucleotide and the target sequence. Under such conditions, the oligonucleotide can form a stable duplex only with a target sequence. Thus, the use of target-specific primers under suitably stringent reverse transcription conditions enables the specific reverse transcription of those target sequences which contain the target primer binding sites. The use of sequence-specific reverse transcription conditions enables the specific reverse transcription of those target sequences which contain the exactly complementary primer binding sites.

The term “antisense strand” refers to the strand of a double stranded DNA molecule which is transcribed into mRNA during transcription. The term “sense strand” refers to the strand of a double stranded molecule which is not transcribed into mRNA during transcription.

The term “non-specific reverse transcription” refers generally to the reverse transcription of nucleic acid sequences other than the target sequence which results from primers hybridizing to sequences other than the target sequence and then serving as a substrate for primer extension. The hybridization of a primer to a non-target sequence is referred to as “non-specific hybridization”, and can occur during the lower temperature, reduced stringency pre-reaction conditions.

The term “reverse transcriptase enzyme” refers generally to an enzyme that has RNA-dependent DNA polymerase activity, namely an enzyme which can utilize an RNA template to incorporate dNTP starting at the 3′OH of an annealed primer sequence. Although retroviral reverse transcriptase enzymes are commonly appreciated by those skilled in the art, it is to be understood that reverse transcriptase enzymes may also be isolated from non-retroviral sources. Examples reverse transcriptase enzymes that can be isolated from non-retroviral sources include mobile genetic elements such as the LTR and non-LRT retrotransposons, among others. The term “reverse transcriptase” can also refer to telomerase enzymes which use RNA to template DNA synthesis at the ends of chromosomes to form telomeres.

A reverse transcriptase enzyme may also have the property of thermostability. The thermostable reverse transcriptase enzymes can withstand the high temperature incubation used to remove the modifier groups, typically greater than 40° C., without suffering an irreversible loss of activity. Modified reverse transcriptase enzymes usable in the methods of the present invention include thermostable reverse transcriptase enzymes as well as thermostable DNA polymerases with substantial reverse transcriptase activity. Thermostable DNA polymerase enzymes with substantial reverse transcriptase activity are known to those skilled in the art, and include the rTth and RQ1 DNA polymerases, among others.

The term “reversibly inactivated”, as used herein, refers generally to an enzyme which has been inactivated by reaction with a compound which results in the covalent modification (also referred to as chemical modification) of the enzyme, wherein the modifier compound is removable under appropriate conditions. The reaction which results in the removal of the modifier compound need not be the reverse of the modification reaction. As long as there is a reaction which results in removal of the modifier compound and restoration of enzyme function, the enzyme is considered to be reversibly inactivated.

The term “reaction mixture” refers to a solution containing reagents necessary to carry out a given reaction.

A “reverse transcription reaction mixture”, refers generally to a solution containing reagents necessary to carry out a reverse transcription reaction, and typically contains oligonucleotide primers and a reverse transcriptase enzyme in a suitable buffer. A reaction mixture is referred to as complete if it contains all reagents necessary to enable the reaction, and incomplete if it contains only a subset of the necessary reagents.

It will be understood by one of skill in the art that reaction components are routinely stored as separate solutions, each containing a subset of the total components, for reasons of convenience, storage stability, and to allow for independent adjustment of the concentrations of the components depending on the application, and, furthermore, that reaction components are combined prior to the reaction to create a complete reaction mixture.

The term “non-activating conditions” refers generally to conditions, for instance of pH and/or temperature, under which the activity of a modified enzyme as described herein has substantially reduced or undetectable activity.

The activity of an enzyme is “substantially reduced” if a modified form of the enzyme has an activity which is reduced by at least 50%, 60%, 70%, 80%, 90% or more (and percentages in between) as compared to the unmodified enzyme.

The term “no substantial increase” in reverse transcriptase activity refers generally to no more than a 0, 5%, 10%, 20%, 30%, 40%, or 50% (and percentages in between) increase in reverse transcriptase activity upon incubation for a particular amount of time under non-activating conditions. In exemplary embodiments, “no substantial increase” in activity refers to undetectable activity upon incubation for a particular amount of time under non-activating conditions.

The term “essentially complete inactivation” of enzyme activity refers generally to a level of activity of a modified enzyme which is at least 20% or less of the unmodified enzyme under non-activating conditions. In exemplary embodiments, “essentially complete inactivation” refers to undetectable activity under non-activating conditions.

### III. General Methods and Compositions

The methods of the present invention involve carrying out a reverse transcription reaction using a chemically modified inactive reverse transcriptase enzyme that can be heat-activated. The modified reverse transcriptase enzyme is substantially inactive at lower temperature and does not does not support primer extension or the formation of extension products, non-specific or otherwise, prior to exposure to incubation at an increased temperature, which activates the reverse transcriptase enzyme. Following the increased temperature incubation which reactivates the enzyme, the reverse transcriptase reaction is maintained at elevated temperatures, which helps to insure reaction specificity. In the methods of the present invention, only the heat-activated enzyme has substantial ability to catalyze the primer extension reaction. Thus, primer extension products are formed only under conditions which enhance reverse transcription specificity.

The reversibly inactivated reverse transcriptase enzymes of the present invention are produced by a reaction between the enzyme and a modifier reagent, which results in a reversible chemical modification of the enzyme, which leads to a substantial reduction or non-detectable enzymatic activity under non-activating conditions. The modification consists of the covalent attachment of the modifier group to the protein. The modifier compound is chosen such that the modification is reversible by incubation at an elevated temperature in the reverse transcription reaction buffer. The modifier is also chosen for compatibility with the integrity of RNA. Suitable enzymes and modifier groups are described below.

### IV. Retroviral Reverse Transcriptase Enzymes

Reverse transcriptase enzymes suitable for the practice of the present invention are well known in the art and can be derived from a number of sources. Three prototypical forms of retroviral reverse transcriptase have been studied thoroughly, and are discussed below for exemplary purposes.

Moloney Murine Leukemia Virus (M-MLV) reverse transcriptase contains a single subunit of 78 kDa with RNA-dependent DNA polymerase and RNase H activity. This enzyme has been cloned and expressed in a fully active form in E. coli (reviewed in Prasad, V. R., Reverse Transcriptase, Cold Spring Harbor, N.Y.: Cold Spring Harbor Laboratory Press, p. 135 (1993)).

Human Immunodeficiency Virus (HIV) reverse transcriptase is a heterodimer of p66 and p51 subunits in which the smaller subunit is derived from the larger subunit by proteolytic cleavage. The p66 subunit has both a RNA-dependent DNA polymerase and an RNase H domain, while the p51 subunit has only a DNA polymerase domain. Active HIV p66/p51 reverse transcriptase has also been cloned and expressed successfully in a number of expression hosts, including E. coli (reviewed in Le Grice, S. F. J., Reverse Transcriptase, Cold Spring Harbor, N.Y.: Cold Spring Harbor Laboratory press, p. 163 (1993)). Within the HIV p66/p51 heterodimer, the 51-kD subunit is catalytically inactive, and the 66-kD subunit has both DNA polymerase and RNase H activity (Le Grice, S. F. J., el al., EMBO Journal 10:3905 (1991); Hostomsky, Z., et al., J. Virol. 66:3179 (1992)).

Members of the Avian Sarcoma-Leukosis Virus (ASLV) reverse transcriptase family are also a heterodimers of two subunits, alpha (approximately 62 kDa) and beta (approximately 94 kDa), in which the alpha subunit is derived from the beta subunit by proteolytic cleavage (reviewed in Prasad, V. R., Reverse Transcriptase, Cold Spring Harbor, N.Y.: Cold Spring Harbor Laboratory Press (1993), p. 135). Members of this family include, but are not limited to, Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase, among others.

ASLV reverse transcriptase can exist in two additional catalytically active structural forms, Ad and a (Hizi, A. and Joklik, W. K., J. Biol. Chem. 252: 2281 (1977)).

Sedimentation analysis suggests the presence of alpha/beta and beta/beta are dimers and that the a form exists in an equilibrium between monomeric and dimeric forms (Grandgenett, D. P., et al., Proc. Nat. Acad. Sci. USA 70:230 (1973); Hizi, A. and Joklik, W. K., J. Biol. Chem. 252:2281 (1977); and Soltis, D. A. and Skalka, A. M., Proc. Nat. Acad. Sci. USA 85:3372 (1988)). The ASLV alpha/beta and beta/beta reverse transcriptases are the only known examples of retroviral reverse transcriptase that include three different activities in the same protein complex: DNA polymerase, RNase H, and DNA endonuclease (integrase) activities (reviewed in Skalka, A. M., Reverse Transcriptase, Cold Spring Harbor, N.Y.: Cold Spring Harbor Laboratory Press (1993), p. 193). The a form lacks the integrase domain and activity.

Various forms of the individual subunits of ASLV reverse transcriptase have been cloned and expressed. These include a 98-kDa precursor polypeptide that is normally processed proteolytically to beta and a 4 kDa polypeptide removed from the beta carboxy end (Alexander, F., et al., J. Virol. 61:534 (1987) and Anderson, D. et al., Focus 17:53 (1995)), and the mature beta subunit (Weis, J. H. and Salstrom, J. S., U.S. Pat. No. 4,663,290 (1987); and Soltis, D. A. and Skalka, A. M., Proc. Nat. Acad. Sci. USA 85:3372 (1988)). (See also Werner S. and Wohrl B. M., Eur. J. Biochem. 267:4740-4744 (2000); Werner S. and Wohrl B. M., J. Virol. 74:3245-3252 (2000); Werner S. and Wohrl B. M., J. Biol. Chem. 274:26329-26336 (1999).) Heterodimeric RSV alpha/beta reverse transcriptase has also been purified from E. coli cells expressing a cloned RSV beta gene (Chernov, A. P., et al., Biomed. Sci. 2:49 (1991)).

### V. Reverse Transcriptases of Non-Retroviral Origin

Although retroviral reverse transcriptase enzymes may be isolated from retroviral sources such as those describe above, it is appreciated that reverse transcriptase enzymes may also be isolated from a large number of mobile genetic elements which are not of retroviral origin. Such mobile genetic elements are resident in the genomes of higher order species and play a function role in life cycle of these mobile genetic elements. Mobile genetic elements are known to encode genes for reverse transcriptase enzymes (reviewed in Howard M Temin, Reverse Transcription in the Eukaryotic Genome: Retroviruses. Pararetroviruses, Retrotransposons, and Retrotranscripts, Mol. Biol. Evol. 2(6):455-468). These elements include, but are not limited, to retrotransposons. Retrotransposons include the non-LTR and LTR mobile genetic elements LINES (such as L1) and SINES (such as SVA elements), and Au elements, among others. (Reviewed by Cordaux and Batzer, Nature Reviews, October 2009, volume 10, pp 691-703.)

### VI. Thermostable DNA Polymerases with Reverse Transcriptase Activity

Certain DNA polymerase enzymes possess the ability to use RNA as a template, and as such, have substantial reverse transcriptase activity. Therefore, DNA polymerase enzymes with substantial reverse transcriptase activity may be used in the practice of the present invention. In some cases, a such DNA polymerases are thermostable. Examples of thermostable DNA polymerase enzymes that possess substantial reverse transcriptase activity, include thermostable DNA polymerases isolated from thermophilic eubacteria or archaebacteria comprising species of the genera: Thermus, Thermotoga, Thermococcus, Pyrodictium, Pyrococcus, and Thermosipho, among others. Representative species from which thermostable DNA polymerases possessing substantial reverse transcriptase activity have been derived include: Thermus aquaticus, Thermus thermophilus, Thermotoga maritima, Pyrodictium occultum, Pyrodictium abyssi, and Thermosipho africanus, among others.

The methods of the present invention are not limited to the use of the enzymes exemplified above. Any enzyme described in the literature with reverse transcription activity can be potentially modified as described herein to produce a reversibly inactivated enzyme suitable for use in the present methods. In general, any reverse transcriptase enzyme which can withstand reactivation incubation temperatures without becoming irreversibly inactivated, is a candidate for modification, as described herein, to produce a reversibly inactivated reverse transcriptase enzyme for use in the present methods. One of skill in the art would be able to optimize the modification reaction and reverse transcription reaction conditions for any given enzyme based on the guidance provided herein.

### VII. Modifier Reagents

In exemplary embodiments of the invention, reversible inactivation of a reverse transcriptase enzyme is carried out by reversible blocking of lysine residues by chemical modification of the ε-amino group of lysine residues. Modification of the lysines in the active region of the protein results in inactivation of the protein. Additionally, modification of lysines outside the active region may contribute to the inactivation of the protein through steric interaction or conformational changes. A number of compounds have been described in the literature which react with amino groups in a reversible manner. For example, amino groups have been reversibly modified by trifluoracetylation (see Goldberger and Anfinsen, 1962, Biochemistry 1:410), amidination (see Hunter and Ludwig, 1962, J. Amer. Chem. Soc. 84:3491), maleylation (see Butler et al., 1967, Biochem. J. 103:78), acetoacetylation (see Marzotto et al., 1967, Biochem. Biophys. Res. Commun. 26:517; and Marzotto et al., 1968, Biochim. Biophys. Acta 154:450), tetrafluorosuccinylation (see Braunitzer et al., 1968, Hoppe-Seyler's Z. Physiol. Chem. 349:265), and citraconylation (see Dixon and Perham, 1968, Biochem. J. 109:312-314; and Habeeb and Atassi, 1970, Biochemistry 9(25):4939-4944).

Exemplary reagents for the chemical modification of the ε-amino group of lysine residues are dicarboxylic acid anhydrides, of the general formula:

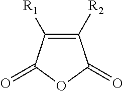

where R1 and R2 are hydrogen or organic radicals, which may be linked, or of the general formula:

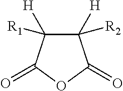

where R1 and R2 are organic radicals, which may be linked, and the hydrogens are cis. The organic radical may be directly attached to the ring by a carbon-carbon bond or through a carbon-heteroatom bond, such as a carbon-oxygen, carbon-nitrogen, or carbon-sulphur bond.

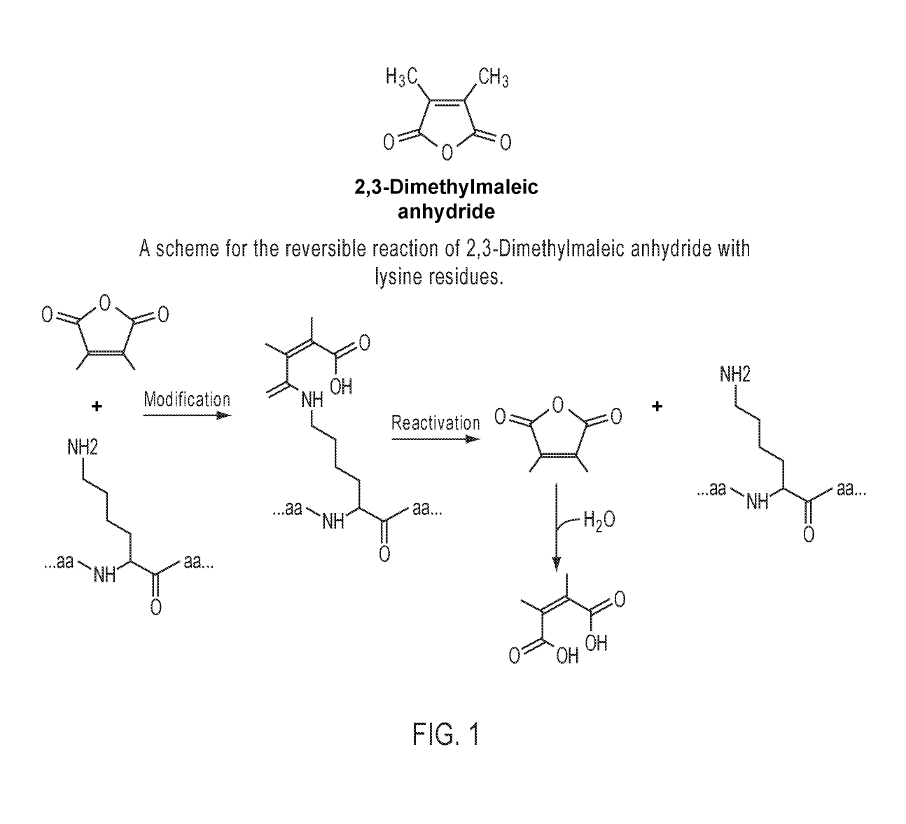

The organic radicals may also be linked to each other to form a ring structure as in, for example, 3,4,5,6-tetrahydrophthalic anhydride. Dicarboxylic acid anhydrides react with the amino groups of proteins to give the corresponding acylated products, as shown herein for 2,3-dimethylmaleic anhydride in FIG. 1.

The reversibility of modifications by the above dicarboxylic acid anhydrides is believed to be enhanced by the presence of either the cis-carbon-carbon double bond or the cis hydrogens, which maintains the terminal carboxyl group of the acylated residues in a spatial orientation suitable for interaction with the amide group, and subsequent deacylation. (See Palacian et al., 1990, Mol. Cell. Biochem. 97:101-111 for descriptions of plausible mechanisms for both the acylation and deacylation reactions.)

Other substituents may similarly limit rotation about the 2,3 bond of the acyl moiety in the acylated product, and such compounds are expected to function in the methods of the present invention. Examples of the exemplary reagents include maleic anhydride; substituted maleic anhydrides such as citraconic anhydride, cis-aconitic anhydride, and 2,3-dimethylmaleic anhydride; exo-cis-3,6-endoxo-Δ⁴-tetrahydropthalic anhydride; and 3,4,5,6-tetrahydrophthalic anhydride. These reagents are commercially available from, for example, Aldrich Chemical Co. (Milwaukee, Wis.), Sigma Chemical Co. (St. Louis, Mo.), or Spectrum Chemical Mfg. Corp. (Gardena, Calif.). Modifications of reverse transcriptase enzymes using the substituted maleic anhydride reagent 2,3-dimethylmaleic anhydride are described in the Examples.

The relative stabilities of the amino groups acylated using the above reagents decreases in the following order: maleic anhydride; exo-cis-3,6-endoxo-Δ⁴-tetrahydropthalic anhydride; citraconic anhydride; 3,4,5,6-tetrahydrophthalic anhydride; cis-aconitic anhydride; and 2,3-dimethylmaleic anhydride (see Palacian et al., supra).

Optimal activation incubation conditions for reverse transcriptase enzymes modified with a particular reagent are determined empirically as described in the Examples. U.S. Pat. No. 5,262,525 describes methods for the chemical modification of proteins which use compounds that are dicarboxylic acid anhydrides prepared by Diels-Alder reaction of maleic anhydride and a diene. Various compounds described in U.S. Pat. No. 5,262,525, which have the stability specified herein may be suitable for use in the present invention.

The methods of the present invention are not limited to the exemplified modifier compounds or to the modification of the protein by chemical modification of lysine residues. Any of the compounds described in the literature which react with proteins to cause the reversible loss of all, or nearly all, of the enzyme activity, wherein the modification is reversible by incubation at an elevated temperature in the reverse transcription reaction buffer, is suitable for preparation of a reversibly inactivated reverse transcriptase enzyme. As new compounds which reversibly modify proteins become available, these too will be suitable for use in the present methods. Thus, compounds for the preparation of the modified reverse transcriptase enzymes of the present invention include compounds which satisfy the following properties: (1) reaction with a reverse transcriptase enzyme which catalyzes primer extension results in a significant inactivation of the enzyme; (2) incubation of the resulting modified enzyme in an aqueous buffer at about pH 7-9 at a temperature at or below about room temperature (25° C.) results in no significant increase in enzyme activity in less than about 20 minutes; and (3) incubation of the resulting modified reverse transcriptase enzyme in a reverse transcription reaction buffer, formulated to about pH 7-9 at room temperature, at an elevated temperature greater than about 40° C., 40° C.-50° C., 50° C.-60° C. or 60° C.-65° C. results in at least a two-fold increase in enzyme activity in less than about 60 minutes. The suitability of a particular modifier compound can be empirically determined following the guidance provided herein. Experimental procedures for measuring the above properties, the degree of attenuation of enzyme activity resulting from modification of the protein and the degree of recovery of enzyme activity following incubation at elevated temperatures in a reverse transcription reaction mixture, are described in the Examples.

### VIII. Preparation of the Reversibly Inactivated Reverse Transcriptase Enzymes

The chemical modification of lysine residues in proteins is based on the ability of the ε-amino group of this residue to react as a nucleophile. The unprotonated amino group is the reactive form, which is favored at alkaline pH. The modification reaction is carried out at pH 8.0 to 9.0 in an aqueous buffer at a temperature at or below room temperature (e.g., 10° C.). The reaction is essentially complete following an incubation for 1-2 hours. Suitable reaction conditions are known in the art and are described further in the Examples. Dicarboxylic acid anhydrides react easily with water to give the corresponding acids. Therefore, a large fraction of the reagent is hydrolyzed during modification of the protein amino groups. The rate of hydrolysis increases with pH. The increase in hydrolysis which occurs at pH greater than about 9 can result in suboptimal acylation of the protein.

In general, a molar excess of the modifier reagent relative to the protein is used in the acylation reaction. The optimal molar ratio of modifier reagent to enzyme depends on the reagent used and can be determined empirically.

As an example, Murine Molony Virus reverse transcriptase is essentially completely inactivated (<5% of original activity) by reaction with a 50-fold or greater molar excess of 2,3-dimethylmaleic anhydride. The minimum molar ratio of modifier which results in essentially complete inactivation of the enzyme can be determined by carrying out inactivation reactions with a dilution series of modifier reagent, as described in the Examples. In the methods of the present invention, it is not necessary that the reverse transcriptase enzyme be completely inactivated, only that the reverse transcriptase enzyme be significantly inactivated.

As used herein, an enzyme is considered to be significantly inactivated if the activity of the enzyme following reaction with the modifier is less than about 50% of the original activity.

A reduction in non-specific reverse transcription can be obtained using a significantly inactivated enzyme. A molar ratio of modifier to enzyme in the reaction can be empirically selected that will result in either essentially complete inactivation or significant inactivation of the enzyme by following the guidance provided herein. Suitable molar ratios for this purpose are provided in the Examples.

Another aspect of the heat-inactivated enzymes of the present invention is their storage stability. In general, the compounds described herein are stable for extended periods of time, which eliminates the need for preparation immediately prior to each use. Reverse transcriptase enzymes modified with reagents such as 2,3-dimethylmaleic anhydride, should be stored refrigerated.

### VIV. Methods for Use of Reversibly Inactivated Reverse Transcription Enzymes

The reverse transcriptase enzymes of the present invention are may be used in reverse transcription reactions to produce cDNA molecules. Optionally, the reverse transcription reaction mixture may additionally contain components necessary for polymerase chain reaction.

The methods of the present invention involve the use of a reaction mixture containing a reversibly inactivated reverse transcriptase enzyme and subjecting the reaction mixture to a high temperature incubation prior to, or as an integral part of, the reverse transcription reaction. The high temperature incubation results in deacylation of modified-amino groups and recovery of enzyme activity. The deacylation of the modified amino groups results from both the increase in temperature and a concomitant decrease in pH. Reverse transcription reactions typically are carried out in a Tris-HCl buffer formulated to a pH of 6.5 to 9.0 at room temperature. At room temperature, the reaction buffer conditions favor the acylated form of the amino group. Although the pH of the reaction buffer is adjusted to a pH of 6.5.0 to 9.0 at room temperature, the pH of a Tris-HCl reaction buffer decreases with increasing temperature. Thus, the pH of the reaction buffer is decreased at the elevated temperatures at which the reverse transcription is carried out and, in particular, at which the activating incubation is carried out. The decrease in pH of the reaction buffer favors deacylation of the amino groups. The change in pH which occurs resulting from the high temperature reaction conditions depends on the buffer used. The temperature dependence of pH for various buffers used in biological reactions is reported, for example, in Good et al., 1966, Biochemistry 5(2):467-477. For Tris buffers, the change in pKa, i.e., the pH at the midpoint of the buffering range, is related to the temperature as follows: Δ pKa/° C.=−0.031. For example, a Tris-HCl buffer assembled at 25° C. undergoes a drop in pKa of 2.17 when raised to 95° C. for the activating incubation. Although reverse transcription reactions are typically carried out in a Tris-HCl buffer, reverse transcription reactions may be carried out in buffers which exhibit a smaller or greater change of pH with temperature. Depending on the buffer used, a more or less stable modified enzyme may be desirable. For example, using a modifying reagent which results in a less stable modified enzyme allows for recovery of sufficient reverse transcriptase enzyme activity under smaller changes of buffer pH.

As disclosed herein, an empirical comparison of the relative stabilities of enzymes modified with various reagents can guide selection of a modified enzyme suitable for use in particular buffers

In general, the length of incubation required to recover enzyme activity depends on the temperature and pH of the reaction mixture and on the stability of the acylated amino groups of the enzyme, which, in turn, depends on the modifier reagent used in the preparation of the modified enzyme. A wide range of incubation conditions are usable; optimal conditions can be determined empirically for each reaction. In general, an incubation is carried out in the reverse transcription reaction buffer at a temperature greater than about 40° C. for between about 10 minutes and about 60 minutes. Optimization of incubation conditions for the reactivation of reverse transcriptase enzymes or for reaction mixtures not specified herein can be determined by routine experimentation following the guidance provided herein.

In an exemplary embodiment, a reverse transcription reaction is carried out using a reversibly inactivated reverse transcriptase enzyme. The annealing temperature used in a reverse transcriptase reaction typically is about 42-65° C., and the reactivation incubation is carried out at a temperature equal to or higher than the annealing temperature, The reverse transcription reaction mixture preferably is incubated at about 37° C.-65° C., 40° C.-50° C., 50° C.-60° C. or 60° C.-65° C. for up to between 3 minutes and about 60 minutes to reactivate the reverse transcriptase enzyme. Suitable reaction incubation conditions for typical activation of modified reverse transcriptase are described in the Examples.

In an exemplary embodiment of the invention, the modified reverse transcription enzyme and initial activation conditions are chosen such that only a fraction of the recoverable enzyme activity is recovered during the initial incubation step.

Subsequently increasing the length of the incubation period will increase the recovery of the reverse transcriptase activity. It is known that an excess of reverse transcriptase enzymes contributes to a non-specific reverse transcription reaction. An advantage of the methods of the present invention is that the methods require no manipulation of the reaction mixture following the initial preparation of the reaction mixture. Thus, the methods are ideal for use in automated reverse transcription systems and with in-situ reverse transcription methods, wherein the addition of reagents after the initial denaturation step or the use of wax barriers is inconvenient or impractical.

The methods of the present invention are particularly suitable for the reduction of non-specific reverse transcription in a reverse transcription reaction. However, the invention is not restricted to any particular reverse transcription system. The reversibly-inactivated enzymes of the present invention can be used in any primer-based reverse transcription system which uses reverse transcriptase enzymes and relies on reaction temperature to achieve reverse transcription specificity.

The present invention also relates to kits, multicontainer units comprising useful components for practicing the present method. A useful kit contains a reversibly-inactivated reverse transcriptase enzyme and one or more reagents for carrying out a reverse transcription and optionally an amplification reaction, such as oligonucleotide primers, substrate nucleoside triphosphates, cofactors, and an appropriate buffer.

The examples of the present invention presented below are provided only for illustrative purposes and not to limit the scope of the invention.

### EXAMPLES

### Example 1

### Modification of Reverse Transcriptase Enzymes with 2,3 Dimethyl Maleic Anhydride

This example describes the modification of SuperScript® III reverse transcriptase using 2,3-dimethylmaleic anhydride. This example generally illustrates a method for the modification of a reverse transcriptase enzyme may by manipulating the molar ratio of the modifier reagent to the reverse transcriptase enzyme to be modified.

Measurements were taken of the activity of the SuperScript® III reverse transcriptase modified by 2,3-dimethylmaleic anhydride to determine the molar ratio of modifier to enzyme required in the inactivation reaction to obtain complete inactivation of DNA polymerase activity as described Example 4, below. SuperScript® III reverse transcriptase (Life Technologies Perkin Elmer, Norwalk Conn.) was used at an initial concentration of 2.0 mg/ml. In the initial experiments, the SuperScript® III reverse transcriptase was purified by heparin chromatography in a Tris/HCl buffer at a pH of 8.5 and modified using various molar ratios of enzyme to 2,3-dimethylmaleic anhydride. A solution of 50 mg of solid 2,3-dimethylmaleic anhydride which is commercially available (Sigma Aldrich Milwaukee, Wis.) was diluted in DMF (N,N di methyl formamide). For one set of modification reactions, a dilution series of the 2,3-dimethylmaleic anhydride solution was created by repeated 2-fold dilutions in DMF after an initial dilution of the stock solution to a concentration of 5.5 mg/ml. For each dilution series, 2,3-dimethylmaleic anhydride solution was added to 100 μl of 2.0 mg/ml SuperScript® III reverse transcriptase, resulting in solutions containing a series of molar ratios of series 2,3-dimethylmaleic anhydride to SuperScript® III reverse transcriptase of approximately 200/1, 100/1, 50/1 and 25/1. Solutions were incubated at 4° C. for 1 hour to inactivate the SuperScript® III reverse transcriptase. A schematic diagram illustrating the modification of lysine residues is presented in FIG. 1. As used herein, a reverse transcriptase enzyme which has been modified in a reaction with an N-fold molar excess of modifier is referred to as an N× enzyme. Thus, the resulting 2,3-dimethylmaleic anhydride modified reverse transcriptase enzymes are referred to herein as 400×, 200×, 100×, 50×, and 25× modified reverse transcriptase enzymes.

### Example 2

### Measurement of in-Activation of Thermostable Reverse Transcriptase, RQ1

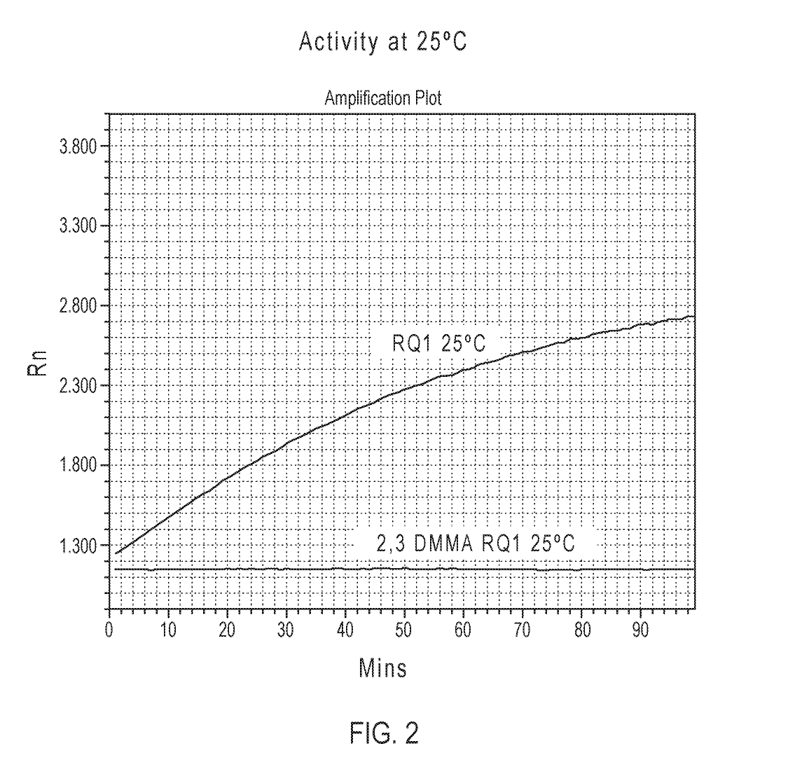

RQ1 reverse transcriptase was subjected to inactivation reactions with 2,3-dimethylmaleic anhydride as described above. As shown in FIG. 2, at 25° C., 2,3-dimethylmaleic anhydride-modified RQ1-RT has no detectable polymerase activity for a period of 90 minutes. Unmodified control enzyme RQ1 demonstrates activity at 25° C.

### Example 3

### The Effect of Increasing Levels of 2,3-Dimethylmaleic Anhydride Modification on RQ1 Reverse Transcriptase Activity

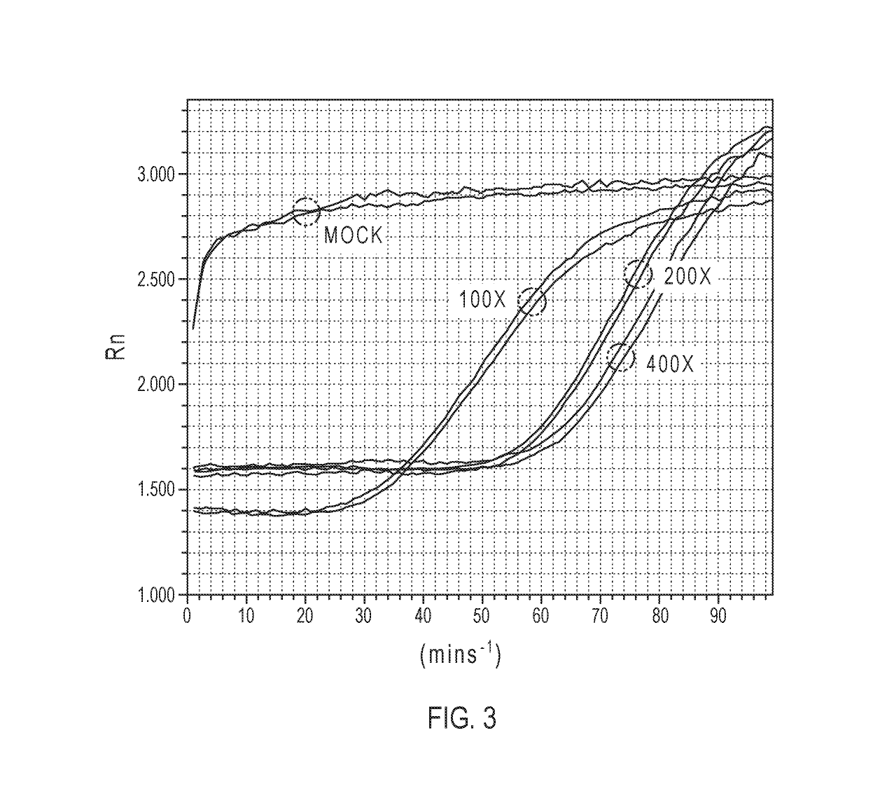

This experiment demonstrates that RQ1-RT modified with increasing molar excesses of 2,3-dimethylmaleic anhydride showed delayed activation. Increasing molar ratios of modifier reagent to enzyme were used to modify thermostable RQ1 reverse transcriptase as described in Example 1. 100× modified RQ1 reactivated earlier than 200× modified RQ1. Although 400× modified RQ1-RT activated later than 100× and 200× modified RQ1, it may be beneficial to delay activation RQ1 reverse transcriptase activity. As shown in FIG. 3, these results demonstrate that RQ1 may be differentially modified according the molar excess of the modification reagent.

### Example 4

### Real Time PCR Assay to Determine the Specificity of Reverse Transcription by Modified Reverse Transcriptase Enzymes Using a TaqMan® Assay

To demonstrate the utility of 2,3-dimethylmaleic anhydride modified-RQ1-RT for use in one step RT-PCR, the specificity of the RT-PCR reverse transcription step was measured using a TaqMan® primer/probe assay that targets an RNA sequence. The RNA sequence used in these experiments is designated the Xeno RNA sequence. The reverse primers, which are responsible for initiating the cDNA from the RNA template, were designed to either be perfectly matched to the RNA sequence or to contain mismatches to the target sequence. The nucleotide sequences of the oligonucleotides used in the assay are as follows.

Perfect match primer (PM):
(SEQ ID NO: 1)
ACCCTTGCTAGTAGGTGTAGATTCTC
Mismatch primer (MM):
(SEQ ID NO: 2)
ACCCTTGCTAGTAGGTGTAGATTCGC
FAM-MGB Probe:
(SEQ ID NO: 3)
ACGTACCAGAGGATCACC
Xeno RNA template sequence:
(SEQ ID NO: 4)
GGGAGAAGAGAATTCGCCCTTGTACTGACGTAAAGTCACTATTTTCGTGC
AACGTACGTCTCGATGTACAACTGCTCTATTACGGTTCATTTTTTTTGTA
GGGTTACGCGGCCAGATGACTCCATCTTATCCCCTTGAAAACATTCTTAT
TTGTACGCCATAGTGGCATCGCGGTTGGATACTAATCGTATTGGACGCAA
GCGCGCTCTACTCAGTTTATAAGACCGCCAACTATTTTCGCAAGATCAGT
GTATTTACGCTGACTCCAGTGGTGAAACTCCTAAGATCTGTTTAGCTATT
GCGCCGTGCGTTTATCAAATCGGGCTTCCCAACATTCATTCTTAGAAGGA
AGCTCGATAGTTCAGAGCTGCGGAAGGCCCAATTTCATATTATATGTATG
AGCCTGTCAATACCTGCACCCACGAACACCACAGTGACTAGAGTATGAGA
GGTCGACGATCTACGGATGGTGATGAGCACGGAGATCTAAGCGTGGAAGT
GGCTATATAGAGCAGATATATTATATGACGTACCAGAGGATCACCTACTA
AAAGACTTTTCGAGAATCTACACCTACTAGCAAGGGTAGCCGATTAGTGG
ATCATCTAAGACATCAAGGCTCAAACTAATTTTACCATGGACGCTGCATT
TACGCTTGCACATTTTATGTTGGCAGCCTTTGCCGCGGCACATAGCGATA
TCCCGTACCCGCTTTTCTTTAAGTTAATCGCCGATGATTGGCTCAATAAT
CGCCTCACTTGTGCGATGACTAGCCAGGCGTTTCCCGCGTTTCTAGATAT
TATCGCGCTTATATAGTATAGACGAGTACCCTTTGTTGTTATTGCAGCAC
CCAACAGAACTAAGTAATCTTTAGGCTGCGGCCGCTTAGGTGGCAGAAGA
TTTGCTCGATGTTCTCAAGTAAAGGACGTCGGGGAGTTGACGGTTGGCAG
GTAACGTATGGATCTTTAATATAATCTAGGCAACAAGTAAGGGCCATTGA
GCGCTTATATGCCGCAGTCTAAAAAAAAAAAAAAAAAAAAAAAAAAAAAA
ACATGAGGATTACCCATGTA

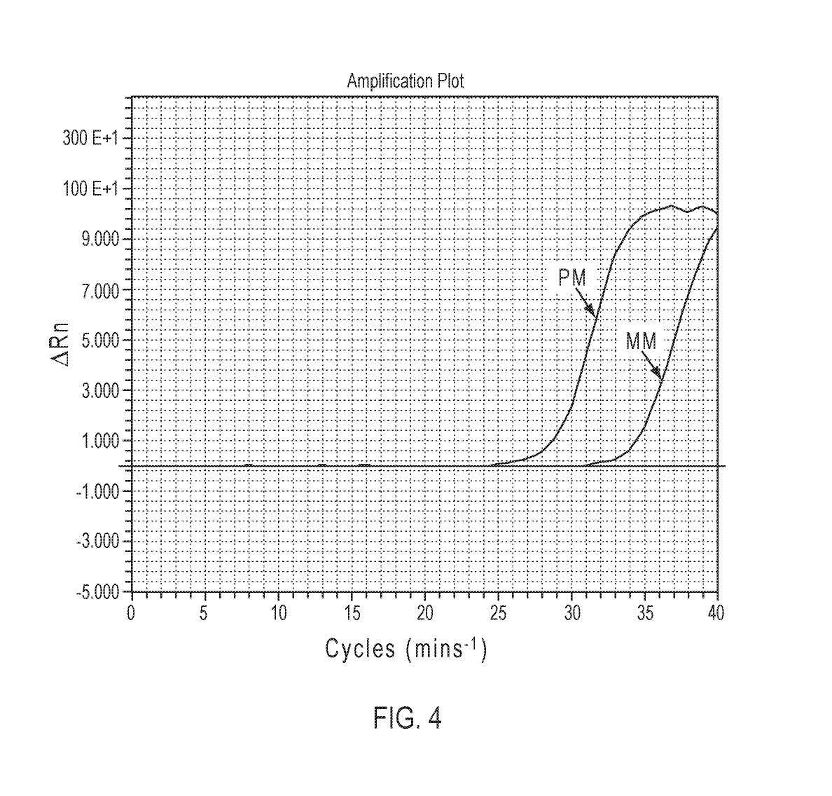

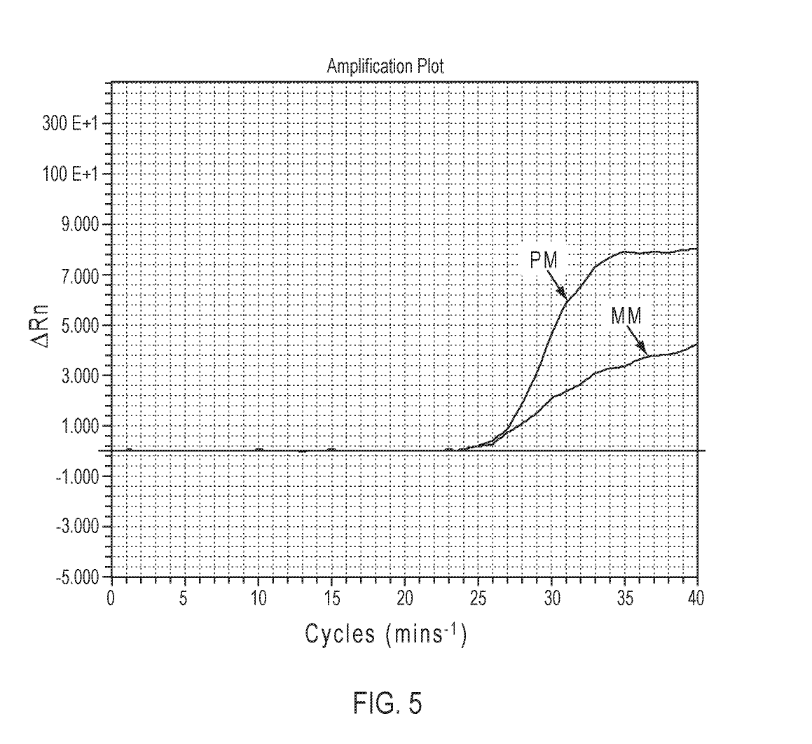

The above TaqMan® reagents were used to measure the specificity of the reverse transcription step in an RT-PCR reaction using 200× modified RQ1-RT. As shown in FIG. 4, RQ1-RT activates at a higher temperature, preventing non-specific extension of the mismatched primer sequence. Our results show that 200×2,3-dimethylmaleic anhydride-modified RQ1-RT can distinguish between a perfectly matched reverse transcriptase primer and a similar primer that contains a single base mismatch. The mismatched primer (MM) shows a 6 Ct delay when compared with the perfectly matched primer (PM). In contrast, the unmodified control enzyme, as shown in FIG. 5, does not distinguish between the perfectly match primer (PM) and the mismatch primer (MM). These results indicate that 2,3-dimethylmaleic anhydride-inactivated RQ1-RT does not efficiently extend the mismatched primer sequence, thus indicating that 200× modified reverse transcriptase was able to distinguish between a perfectly matched and a mismatch reverse primer sequence in a reverse transcription reaction. Other levels of enzyme modification also provide specificity of reverse transcriptase.

### Example 5

### Real Time PCR Assay to Determine the Activity of Modified SuperScript® III Using a DNA-Binding Dye Assay

Measurements of polymerase activity for non-thermostable reverse transcriptase enzymes were conducted using a SYBR Green I assay essentially as described in Nucleic Acids Res. 2004; 32(3): 1197-1207. An oligo dT/polyA substrate was prepared by annealing 40 ul of an oligo dT primer at a concentration of 7.1 μM to 1 ml of a 1 mg/ml poly A solution in water. Samples of modified SuperScript® III reverse transcriptase enzymes were reactivated in reaction buffer by treatment at 50° C. for between 0 and 60 minutes. A 20 μl volume of reactivated enzyme in 1× reactivation buffer was added to 80 μl of a 1× reaction mix which comprised a final dTTP concentration 2.5 μM and a substrate concentration 0.25 ug/ml and 10 mM DTT. The reverse transcription reaction was initiated at 25° C. and a reaction time course was conducted. Reaction samples (5 μl) were taken at various time points and added to a 1:200 dilution of PicoGreen (Molecular Probes) in TE (10 mM Tris-HCl pH 8.0 and 1.0 mM EDTA pH 8.0). The amount of cDNA synthesized was quantified with a Spectromax M5 Spectrofluorometer (Molecular Devices). The amount of cDNA synthesized was measured by determining the fluorescence of SYBR Green 1 which preferentially binds to double stranded DNA or RNA/DNA hybrid strands. The activity of the reactivated SuperScript® III reverse transcriptase was determined by comparing the initial rate of the reactivated enzyme with that of the unmodified control SuperScript® III reverse transcriptase (Life Technologies).

### Example 6

### Inactivation and Heat Recovery of Reverse Transcriptase Activity Using Enzyme Modified with 2,3-Dimethylmaleic Anhydride

This example describes activity measurements of the resulting 2,3-dimethylmaleic anhydride-modified SuperScript® III reverse transcriptase of Example 2, using the reverse transcriptase activity assay as described in Example 2, before and after re-activation of the modified enzyme by heat incubation. Samples of 2,3-dimethylmaleic anhydride modified SuperScript® III reverse transcriptase were diluted 2.5/20 in a buffer containing of 25 mM Tris-HCl, 75 mM KCl, 5 mM MgCl₂, and 10 mM DTT. The buffer pH was 7.25 at room temperature. Diluted samples of 2,3-dimethylmaleic anhydride-modified SuperScript® III reverse transcriptase were incubated at 50° C. for 40 minutes or maintained on ice to provide a control activity reference. Following heat treatment, samples were assayed for activity as described in Example 1. The reverse transcriptase activities following treatment are shown below. The molar ratios refer to the molar ratio of 2,3-dimethylmaleic anhydride to SuperScript® III reverse transcriptase used in the modification reactions.

TABLE 1

| | Activity (% of control) | Activity (% of control) |
|---|---|---|
| Molar ratio | Unheated | 50° C. incubation |
| Control | 100 | |
| 100X | 0 | 80 |
| 50X | 0 | 90 |
| 25X | <15 | 95 |

As shown in Table 1, complete inactivation of SuperScript® III reverse transcriptase was obtained using greater than 25-fold molar excesses of 2,3-dimethylmaleic anhydride to enzyme. Following incubation of the completely inactivated SuperScript® III reverse transcriptase at 50° C. for 40 minutes, a minimum of 80% of the activity was recovered. Although more enzyme activity was recovered using the 50× modified 2,3-dimethyl maleic anhydride SuperScript® III reverse transcriptase as compared with the 100× modified enzyme, it may be more practical to use the 100× (or higher) 2,3-dimethylmaleic anhydride modified SuperScript® III reverse transcriptase in a commercial kit to allow for greater manufacturing tolerances.

### Example 7

### The Effect of Time of Activation on Recoverable Activity of 100×2,3-Dimethylmaleic Anhydride Modified SuperScript® III Reverse Transcriptase

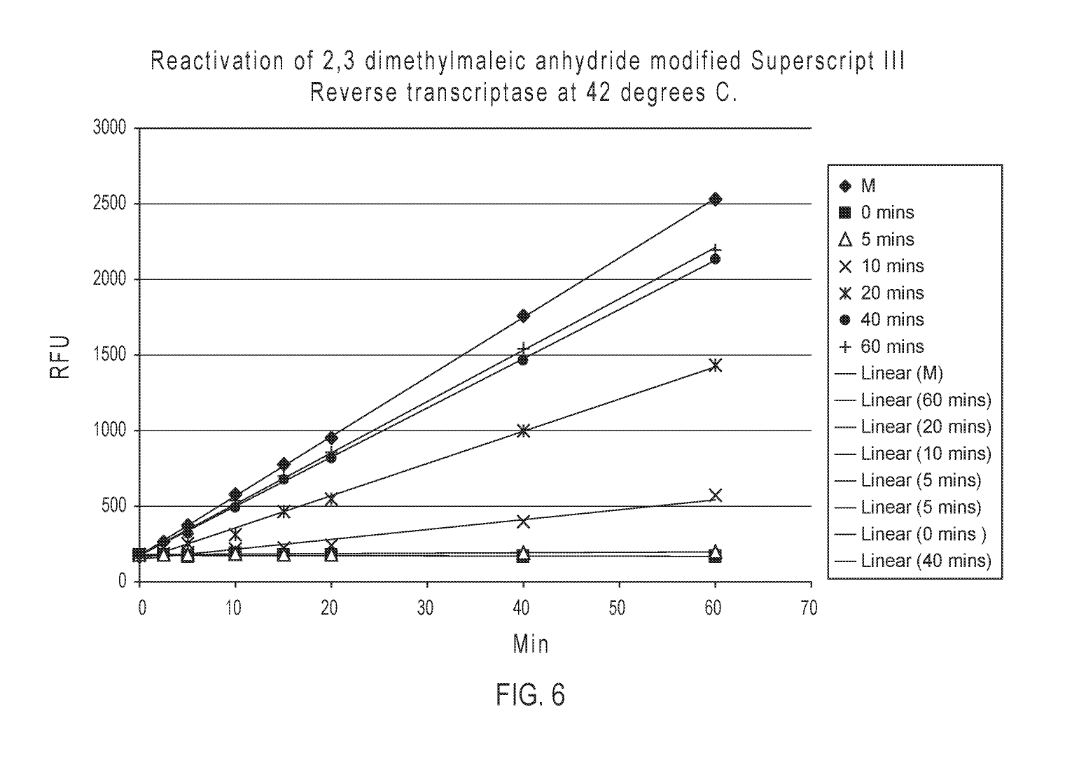

Samples of 100× modified 2,3-dimethylmaleic anhydride modified SuperScript® III reverse transcriptase were diluted 2.5/20 in a buffer containing 25 mM Tris-HCl, 75 mM KCl, 5 mM MgCl₂, and 10 mM DTT. A time course of reactivation was established by removing 2.5 μl samples of 100× modified 2,3-dimethylmaleic anhydride-modified SuperScript® III reverse transcriptase at time intervals of 0, 5, 10, 20, 40, and 60 minutes of heat treatment at 42° C. After the timed heat treatments, each sample of 100× modified 2,3-dimethylmaleic anhydride-modified SuperScript® III reverse transcriptase was returned to ice to prevent further activation. A sample was retained on ice to act as a reference control. The amount of recovered activity was measured using the assay described in Example 2. The amount of activity recovered at each time point is presented in Table 2 and shown graphically in FIG. 6.

TABLE 2

| Time | % Activity of Control |
|---|---|
| 0 | 0% |
| 5 | <1% |
| 10 | 16% |
| 20 | 54% |
| 40 | 82% |
| 60 | 86% |

Substantial reactivation of 100×2,3-dimethylmaleic anhydride-modified SuperScript® III reverse transcriptase was observed within 15 minutes of activation. Higher amounts of activity were obtained after longer incubation periods. Although substantial enzyme activity was recovered within 10 minutes of incubation at 42° C. using the 100× modified enzyme, the use of higher temperatures can be used to promote faster rates of reactivation. While 100× modified SuperScript® was used in this experiment, SuperScript® III reverse transcriptase modified with other molar ratios of 2,3-dimethylmaleic anhydride can also be used, thus allowing activation at slower or faster rates under the same reactivation temperature by using higher or lower ratios for modification. Different modification ratios of SuperScript® III reverse transcriptase will be practical for use in a commercial kit to permit flexible hot start reactivation of the SuperScript® III reverse transcriptase

### Example 8

### Sensitivity, Dynamic Range, Selectivity and Accuracy of Strand-Specific Assays

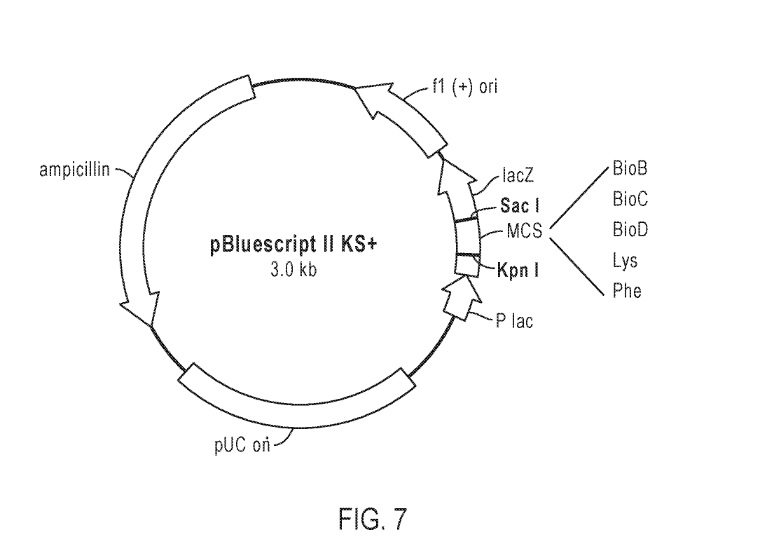

These experiments demonstrate that RQ1-RT modified with 2,3-dimethylmaleic anhydride may be used to accurately quantitate sense and antisense mRNA transcripts. The T7 and T3 promoters of the pBluescript II KS+ plasmid shown in FIG. 7 were used to generate sense and antisense mRNA transcripts which were then assayed in TaqMan® assays using chemically modified RQ1-RT (SSTAY), and other commercially available reverse transcriptases.

Five bacterial gene clones, BioB, BioC, BioD, Lys and Phe, in plasmid pBluescript II KS+ were purchased from the American Type Culture Collection. Sense and antisense transcripts of each gene were generated by in vitro transcription using the MEGAscript® T7 Kit and MEGAscript® T3 Kit (Ambion), respectively. All in vitro transcribed RNA transcripts were further purified by RNeasy Mini Kit (Qiagen) and quantified by Nanodrop (Thermo Scientific). The integrity of these transcripts was also confirmed by Bioanalyzer analysis (Agilent). The sense and anti-sense bacterial RNA pools were constructed by combining equal copies of each transcript at 5×10⁹ copy/μl concentration with 10 ng/μl UHR (Stratagene) as background. Strand-specific reverse transcription reactions were performed in 20 μl volume with Reverse Transcriptase Buffer (10 mM Tris-HCl, 90 mM KCl, pH 8.6), 0.2 mM dNTP, 0.25 U/μl HS-RQ1, 1 mM MgCl₂, 0.75 M strand-specific RT primers and series dilutions of sense or anti-sense RNA pools. The RT reactions were carried out at 62° C. for 30 min, the cDNA products were then diluted 20 fold in PCR reactions with Chelating Buffer (5% glycerol, 10 mM tris/HCl pH 8.6, 100 mM KCl, 0.05% tween 20, 0.75 mM EGTA) and 2× Gene Expression TaqMan® Master Mix (Life Technologies) and specific TaqMan® primers and probes for each individual target. Quantitative real-time PCR was performed using Applied Biosystems 7900HT system with 4 PCR replicates for each target. Strand-specific reverse transcription reactions using other commercially available RT enzymes were performed following manufacturer's recommendations with modifications by using strand-specific RT primers in the RT reaction followed by regular real-time PCR using TaqMan® assays.

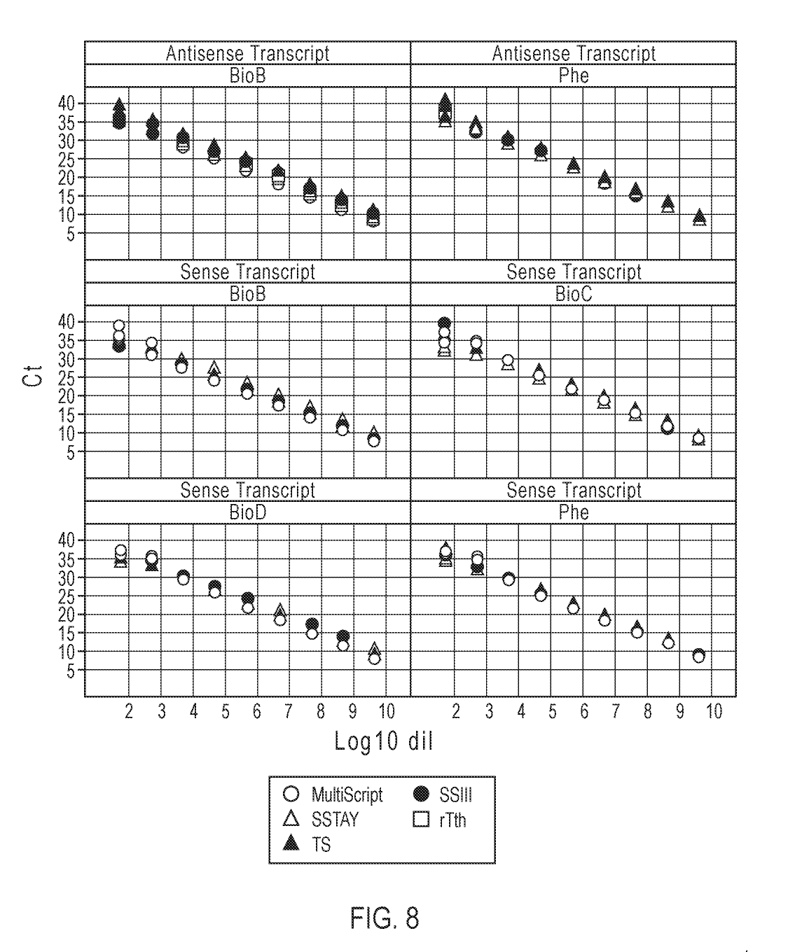

As shown in FIG. 8, this method is able to achieve high sensitivity and a seven log dynamic range.

To demonstrate the improved accuracy of the strand-specific assays using the chemically modified RQ1-RT, the sense transcript was assayed in the presence of varying amounts of antisense transcript. Briefly, different amounts of the anti-sense RNA pool was spiked into the sense RNA pool at a constant concentration (5×10⁵ copy/μl) with different ratios: 1:1, 1:2, 1:5, 1:10, 1:100 and 1:1000. Strand-specific RT-PCR was performed on these samples to determine level of sense and antisense transcripts using HS-RQ1, SuperScript® III or ThermoScript® RT enzymes.

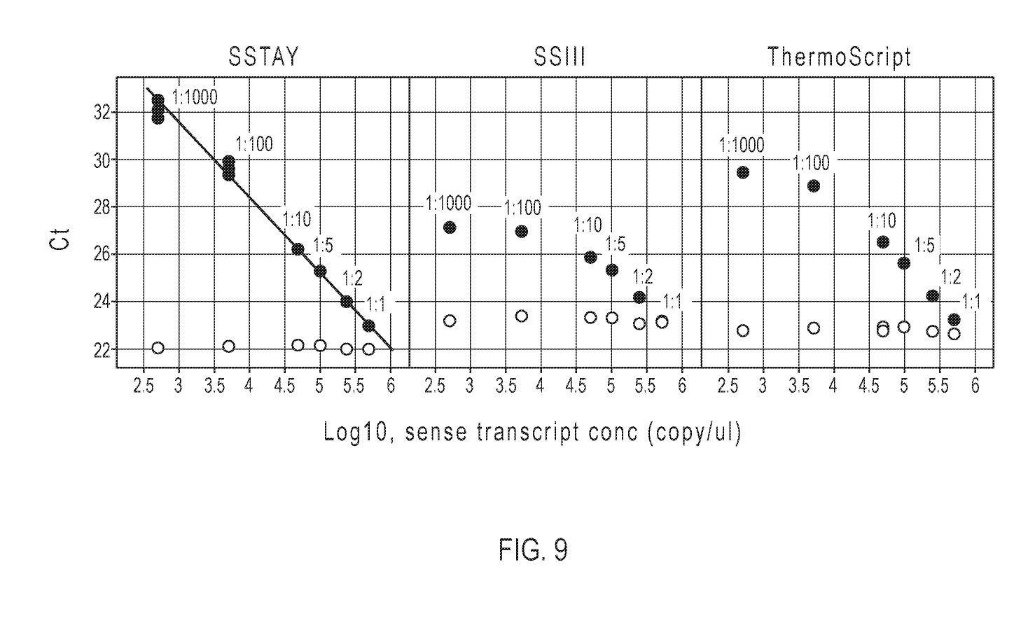

As shown in FIG. 9, use of the modified RQ1-RT allowed a linear response with up to a 1,000 fold excess of the antisense strand. Furthermore, it can accurately determine 1:1, 1:2, 1:5, 1:10, 1:100 and 1:1000-fold difference between sense and antisense transcripts in the same sample. The demonstrated accuracy was far superior to other benchmarked RT enzymes.

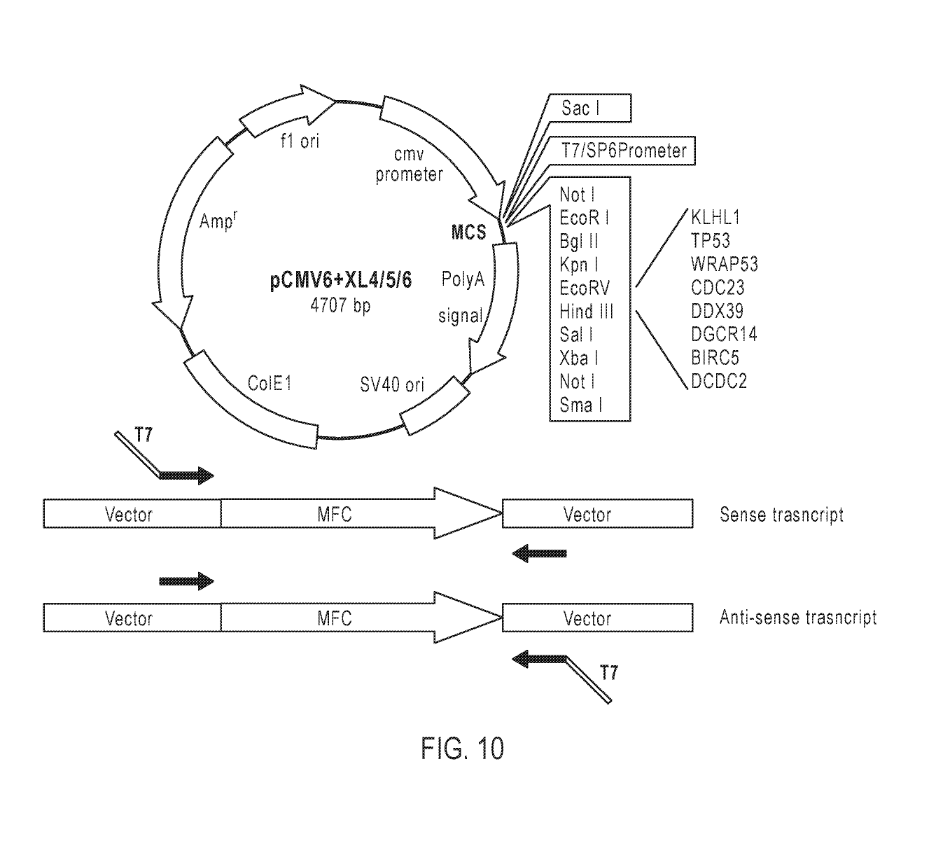

The strand-specific assay may also be used in mammalian transcription systems. FIG. 10 illustrates the pCMV6-XL4/5/6 plasmid which may be used to generate both sense and antisense transcripts. The ability to discriminate between the sense and anti-sense transcripts as measured by the delta Ct (dCt) was compared for four different reverse transcriptases.

The mammalian full length cDNA clones in plasmid pCMV6-XL4/5/6 were purchased from Origene. T7 promoter sequence was introduced to the cDNA inserts by PCR using the following PCR primer pairs:

Sense strand:
(SEQ ID NO: 5)
5′GCGTAATACGACTCACTATAGGGCCGCGAATTCGGCACGAG 3′
(SEQ ID NO: 6)
5′GCGCGCGGCCGCAATCTAGAG 3′
Anti-sense strand:
(SEQ ID NO: 7)
5′GCGGGCCGCGAATTCGGCACGAG 3′
(SEQ ID NO: 8)
5′GCGTAATACGACTCACTATAGGCGCGGCCGCAATCTAGAG 3′

PCR products were checked on Agorose gels and purified using QIAquick PCR Purification Kit (Qiagen). Sense and antisense transcripts of each gene were generated by in vitro transcription using the MEGAscript® T7 Kit (Ambion). All in vitro transcribed RNA transcripts were further purified by RNeasy Mini Kit (Qiagen) and quantified by Nanodrop. The integrity of these transcripts were also confirm by Bioanalyzer analysis (Agilent). The sense and anti-sense mammalian RNA pools were constructed by combining equal copies of each transcript at 5×10⁹ copy/ul concentration with 10 ng/ul yeast RNA (Ambion) as background. The sensitivity of Strand-specific RT-PCR using the modified RQ1-RT was demonstrated by performing strand-specific RT-PCR as described previously using the correct strand RT primers; while the specificity of the method was accessed by performing strand-specific RT-PCR using the opposite strand RT primers. The selectivity of the method can be evaluated as dCt, which is the Ct difference between specific signal (correct strand RT primer) and non-specific signal (opposite strand RT primer).

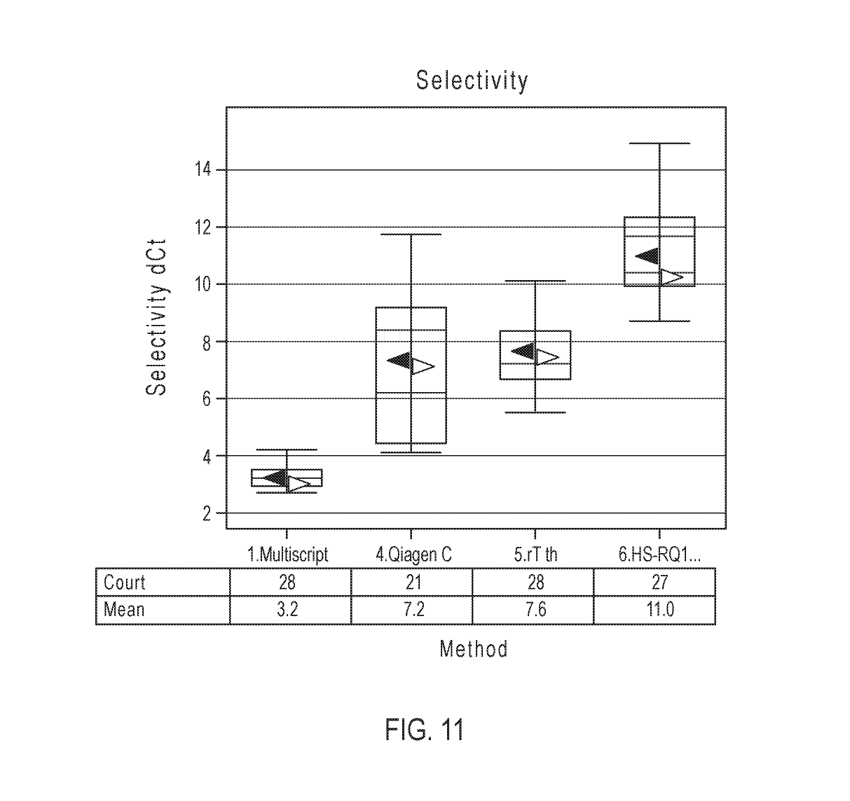

As shown in FIG. 11, the chemically modified RQ1-RT showed a dCt of 11, the highest selectivity of the reverse transcriptases tested.

It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

The section headings used herein are for organizational purposes only and are not to be construed as limiting the subject matter described. All documents, or portions of documents, cited in this application, including but not limited to patents, patent applications, articles, books, and treatises are hereby expressly incorporated by reference in their entirety for any purpose. In the event that one or more of the incorporated documents defines a term that contradicts that term's definition in this application, this application controls.

All references cited herein, including patents, patent applications, papers, text books, and the like, and the references cited therein, to the extent that they are not already, are hereby incorporated by reference in their entirety. In the event that one or more of the incorporated literature and similar materials differs.

1.-30. (canceled)

31. A reverse transcription reaction mixture comprising a modified M-MLV reverse transcriptase having reduced RNase H activity and an aqueous buffer formulated to a pH less than 8 at about 25° C., wherein said reverse transcriptase comprises at least one lysine residue modified by an anhydride selected from the group consisting of 2,3-dimethylmaleic anhydride; maleic anhydride; exo-cis-3,6-endoxo-Δ⁴-tetrahydrophthalic anhydride; citraconic anhydride; 3,4,5,6-tetrahydrophthalic anhydride; and cis-aconitic anhydride, and wherein said reaction mixture is at a temperature less than about 40° C.

32. The reaction mixture of claim 31, wherein the ε-amino group of said at least one lysine residue is blocked.

33. The reaction mixture of claim 31, wherein said at least one lysine residue is located within an active region of said M-MLV reverse transcriptase.

34. The reaction mixture of claim 37, wherein said modification of said at least one lysine residue is reversible.

35. The reaction mixture of claim 34, wherein said modification is reversible at a temperature between about 40° C. to 50° C. in an aqueous buffer that has been formulated to a pH less than 8 at about 25° C.

36. The reaction mixture of claim 31, wherein said chemical agent is covalently linked to said at least one lysine residue.

37. The reaction mixture of claim 31, wherein said reverse transcriptase is at least 50% inactive.

38. The reaction mixture of claim 31, wherein said reverse transcriptase is at least 80% inactive.

39. The reaction mixture of claim 38, wherein said modified reverse transcriptase exhibits increased activity at a temperature between about 40° C. to 50° C. in an aqueous buffer that has been formulated to a pH less than 8 at about 25° C.

40. The reaction mixture of claim 39, wherein said reverse transcriptase exhibits increased activity for up to 60 minutes at a temperature between about 40° C. to 50° C. in an aqueous buffer that has been formulated to a pH less than 8 at about 25° C.

41. A reverse transcription reaction mixture comprising a modified M-MLV reverse transcriptase having reduced RNase H activity and an aqueous buffer formulated to a pH less than 8 at about 25° C., wherein said reverse transcriptase comprises both acylated and deacylated lysine residues, and wherein said reaction mixture is at a temperature between about 40° C. to 65° C.

42. The reaction mixture of claim 41, wherein said composition is at a temperature between about 45° C. to 55° C.

43. The reaction mixture of claim 41, wherein said pH is between 7 to 8.

44. The reaction mixture of claim 41, wherein said reverse transcriptase is at least 50% active.

45. The reaction mixture of claim 41, wherein reverse transcriptase is at least 80% active.

46. The reaction mixture of claim 41, further comprising at least one component selected from the group consisting of an RNA sample, an RNA-specific primer, DTT, a dioxynucleotide (dNTP), and a cDNA.

47. The reaction mixture of claim 41, wherein said modified M-MLV is a mutant M-MLV.

48. The reaction mixture of claim 41, wherein said modified M-MLV exhibits no reverse transcriptase activity at alkaline pH at a temperature at or below about 25° C. for up to 20 minutes.

49. The reaction mixture of claim 41, wherein the number of acylated residues is greater than the number of deacylated residues.


SEQUENCE LISTING (4 sequences)

SEQ ID NO: 1 - DNA, 26 bp, Artificial
  Synthetic
acccttgctagtaggtgtagattctc

SEQ ID NO: 2 - DNA, 26 bp, Artificial
  Synthetic
acccttgctagtaggtgtagattcgc

SEQ ID NO: 3 - DNA, 18 bp, Artificial
  Synthetic
acgtaccagaggatcacc

SEQ ID NO: 4 - DNA, 1070 bp, Artificial
  Synthetic
gggagaagagaattcgcccttgtactgacgtaaagtcactattttcgtgcaacgtacgtctcgatgtacaactgctctattacggttcattttttttgtagggttacgcggccagatgactccatcttatccccttgaaaacattcttatttgtacgccatagtggcatcgcggttggatactaatcgtattggacgcaagcgcgctctactcagtttataagaccgccaactattttcgcaagatcagtgtatttacgctgactccagtggtgaaactcctaagatctgtttagctattgcgccgtgcgtttatcaaatcgggcttcccaacattcattcttagaaggaagctcgatagttcagagctgcggaaggcccaatttcatattatatgtatgagcctgtcaatacctgcacccacgaacaccacagtgactagagtatgagaggtcgacgatctacggatggtgatgagcacggagatctaagcgtggaagtggctatatagagcagatatattatatgacgtaccagaggatcacctactaaaagacttttcgagaatctacacctactagcaagggtagccgattagtggatcatctaagacatcaaggctcaaactaattttaccatggacgctgcatttacgcttgcacattttatgttggcagcctttgccgcggcacatagcgatatcccgtacccgcttttctttaagttaatcgccgatgattggctcaataatcgcctcacttgtgcgatgactagccaggcgtttcccgcgtttctagatattatcgcgcttatatagtatagacgagtaccctttgttgttattgcagcacccaacagaactaagtaatctttaggctgcggccgcttaggtggcagaagatttgctcgatgttctcaagtaaaggacgtcggggagttgacggttggcaggtaacgtatggatctttaatataatctaggcaacaagtaagggccattgagcgcttatatgccgcagtctaaaaaaaaaaaaaaaaaaaaaaaaaaaaaaacatgaggattacccatgta